(19) United States
(12) Patent Application Publication
(10) Pub. No.: US 2025/0099235 A1
(43) Pub. Date: Mar. 27, 2025

(54) PROSTHESIS FOR HEART VALVE AND METHOD FOR CONSTRUCTING A COMPONENT THEREOF

(71) Applicant: Innovheart S.r.l., Milano (MI) (IT)

(72) Inventors: Giovanni RIGHINI, Gland (CH); Gianluca PANE, Ivrea (TO) (IT); Enrico BRESCIANO, Milano (MI) (IT)

(21) Appl. No.: 18/832,638

(22) PCT Filed: Jan. 24, 2023
(86) PCT No.: PCT/IB2023/050574

§ 371 Date: Jul. 24, 2024

(30) Foreign Application Priority Data
Jan. 25, 2022 (IT) 102022000001208

(51) Int. Cl. A61F 2/24

(52) U.S. Cl. CPC A61F 2/2415

(57) ABSTRACT

A prosthesis for a heart valve for implantation via a transcatheter procedure, including an expansible central body, a containment portion with one or more sub-components, and at least one connecting block for connecting each sub-component of the containment portion to the central body. A coupling mechanism of the mechanical type is provided for coupling the at least one connecting block with the expansible central body.

### FIELD OF THE INVENTION

The present invention relates to a prosthesis for a heart valve and a method for constructing a component thereof.

The invention has been developed with particular regard, though in a non-limiting manner, to a valve prosthesis which is intended to replace the physiological function of a poorly working heart valve with particular though non-limiting reference to a heart prosthesis for an atrioventricular heart valve. This prosthesis for a heart valve has been developed specifically for use by means of transcatheter implantation procedures.

### TECHNOLOGICAL BACKGROUND

Heart valves are complex and delicate members which manage the correct function of the human heart. The main objective thereof involves making the haematic flow inside the cardiac cavities unidirectional, being necessary both during the phase of filling the cavities, the diastolic phase, and during the phase of discharging the blood, the systolic phase.

In order to optimize the efficiency of the pumping action of the blood, the heart is structured in two different compartments, the right and left compartments, respectively, each of which is in turn subdivided into two chambers, the atrium and ventricle, respectively. The right compartment of the heart, which is composed of the right atrium and ventricle, brings back the blood from the peripheral circulation and directs it towards pulmonary circulation for the oxygenation thereof. The left compartment, which is similarly subdivided into the left atrium and ventricle, supplies peripheral circulation, bringing back the oxygenated blood from the pulmonary circulation and pumping it towards the systemic circulation.

In order to make the haematic flow unidirectional inside the heart, a valve is positioned at the outlet of each chamber. The valves positioned at the outlet of the atria are called the atrioventricular valves because they connect the atrial chamber to the ventricular chamber of each side of the heart. In the right side of the heart, this valve is also called the tricuspid valve, in the left side it is usually referred to as the mitral valve. Finally, the valve which is positioned at the outlet of the right ventricle is called the pulmonary valve while the valve at the outlet of the left ventricle is called the aortic valve.

The pathologies which alter the function of a heart valve are among the most serious in the cardiovascular field. Among them, insufficiency of the mitral valve, or the inability thereof to completely close, is a valve pathology which is very debilitating because it reduces the efficiency of the pumping action of the left side of the heart, which is responsible for the blood circulation of the entire body.

In the current state of the art, the standard therapy for treating severe valve malfunctions is to replace the valve with an implantable prosthesis. In other cases, mainly in the case of mitral valve dysfunction, a valve repair is carried out. In both cases, an open-heart surgical procedure is carried which out, affords access to the direct malfunctioning valve. This procedure requires the temporary arrest of the heart and the generation, by means of suitable pumps and oxygen exchangers, of an artificial extra-corporeal blood circulation. Notwithstanding the refinement of the techniques for managing the cardiac arrest and the improvement of the extra-corporeal circulation systems, the open-heart therapy involves risks as a result of the invasive nature thereof and the duration of the procedure. In fact, implantable prostheses, both the ones for repair and the ones for replacement, which are usually used in conventional surgery typically require a long operation for being fixed at the implant site by means of specific suturing techniques. In some cases, it is not even possible any surgical therapy as a result of the general conditions of the patient, for example, as a result of the advanced age of the patient or the presence of concomitant pathologies.

to overcome these limitations, interventional procedures with low invasiveness, called transcatheter procedures, have been recently developed. To this end, radially collapsible prostheses which are able to self-secure at the implant site are used. The prostheses can be implanted by means of catheters which can navigate inside the vascular system and release the heart prosthesis by reaching the implantation site from a remote access created, for example, in a peripheral vessel, such as a vein or a femoral artery. The valve dysfunctions can thereby be corrected with the heart still beating and with limited use of surgical practice. At present, the transcatheter techniques are in current clinical use only for the treatment of the aortic valve.

The situation is different with regard to the treatment of the dysfunctions of the atrioventricular valves, in particular for treating mitral insufficiency. The complex anatomical configuration of the valve and the structures which surround it, the variability of the pathologies, which are also very different from each other and which directly or indirectly affect the valve, make it extremely difficult to comply with the requirements for a safe and effective implantation in the mitral valve via the transcatheter route.

In recent years, there have been developed a number of transcatheter prostheses for atrioventricular valves which can be implanted in accordance with different accesses and procedures. A first implantation methodology which is particularly indicated for the mitral valve provides for creating access to the native valve to be replaced through the apex of the left ventricle, which methodology has already been developed by the same Applicant (WO 2014/080338 and WO 2014/080339). The procedure provides for a thoracic incision in order to expose the apex of the left ventricle. Via the apex, a direct access to the ventricular chamber is surgically obtained, sometimes by means of temporary positioning, that is to say, in a manner limited to the duration of the procedure, of an apical port. Via the apical access, from time to time the catheters necessary for carrying out the implantation procedure are subsequently inserted.

Another methodology which has also been developed by the same Applicant (WO 2021/014400) instead provides for transseptal access. The term “transseptal access” is intended to be understood to be the access to the atrioventricular valve to be replaced which, starting from a peripheral femoral vein, ascends the inferior vena cava up to the right atrium. In the case of implantation in a mitral position, it is also necessary to reach the left atrium via an opening which is created, with interventional methods, in the septum between the two atria. The left atrium affords antegrade access to the native mitral valve. In this manner, damage to, that is to say the perforation of, the left ventricle associated with the transapical procedure, which affords access from the ventricular side, that is to say retrograde, to the mitral valve, is prevented.

The valve prostheses implanted via the transcatheter route have been specifically studied to be able to assume a configuration with minimal radial dimensions, in order thereby to be able to be inserted in a catheter for navigating inside the cardiovascular system of the patient. The patent application WO 2015/118464 by the same Applicant describes, for example, a transcatheter prosthesis which is particularly suitable for the purpose. The prosthesis has a support and interface structure with the native valve and a group of flexible prosthetic leaflets which are fixed to the interior thereof. The prosthesis structure particularly comprises a valve-operated central body, a containment portion which is formed by two or more arcuate segments and connecting blocks which are mounted at the end of connecting arms which are fixedly joined to the structure of the central body, in order to provide mechanical continuity between the central body and the arcuate segments.

In the patent application WO 2015/118464, the connecting blocks appear to be fixed to the connecting arms by means of welding or soldering. In fact, it is technologically complex and uneconomical to construct connecting blocks, in particular with a design which satisfies the requirements imposed by the prosthesis described in the patent application WO 2015/118464, in a single piece with the connecting arms of the structure of the central body. In fact, it must be noted that the structural components of this type of prosthesis are usually constructed by laser-cutting from a tube of super-elastic alloy (for example, Nitinol) and this technology does not allow the central body and the connecting blocks to be constructed in a single piece.

However, the Applicant has noted that the known prostheses have a problem of durability of the weld connection between the connecting blocks and the flexible arms because the in-vivo operative conditions and particularly the cardiac cycle generate the application of significant cyclical loads on these elements. At the same time, the consequences of in-vivo structural failures are evidently catastrophic because the separation of the connecting blocks from the connecting arms in the implanted prosthesis could cause a dislodgment or even a migration of the prosthesis with serious adverse outcomes for the patient.

### STATEMENT OF INVENTION

One of the objects of the invention is to solve the problems of the prior art. In particular, it is intended to provide a prosthesis for a heart valve which is collapsible and which is durable. Another object is to provide a prosthesis for a heart valve which minimizes the risk of separation between the connecting blocks and the central body both during the implantation and during use, so as to be particularly safe over the long term also in presence of significant fatigue loads. Another object is to provide a device which is economical and simple to produce.

In order to achieve these objects and other objects, the application relates to a prosthesis according to the appended claims.

There is particularly described a prosthesis for a heart valve for implantation with a transcatheter procedure, comprising an expansible central body, a containment portion having one or more sub-components, and at least one connecting block for connecting each sub-component of the containment portion to the central body.

According to one aspect, each connecting block is provided with a mechanical coupling mechanism with respect to the central body and more specifically to connecting arms which are fixedly joined to the structure of the central body. The coupling mechanism is preferably of the snap-fit type. The coupling mechanism is preferably reversible.

There is thereby obtained a connection which is extremely stable, simple in terms of design and construction and which is not subjected to specific risks of breaking even if subjected to heavy cyclical fatigue loads. Additionally, the solutions described preserve the possibility of constructing the structure of the central body and the connecting arms by means of standard laser-cutting techniques which constitute the consolidated technology for this type of components. Cutting the base material, which is in the form of a tube, by means of laser is in fact the known, preferred process for obtaining the structure of the central body, but it does not allow production of the geometries necessary for the functionality of the connecting block. Therefore, it is technically and economically advantageous to construct the connecting block separately from the structure of the central body. However, a stable and durable connection between these two components is fundamental and is obtained by the present invention.

According to one aspect, the coupling mechanism comprises at least one housing which is provided on the connecting block and in which at least one corresponding coupling member provided on the central body is inserted. Preferably, this coupling member is provided with one or more hooking teeth for coupling in a snap-fitting manner with the connecting block. The coupling member, or the housing on the connecting block, or both the parts of the coupling mechanism have an elastically deformable portion so as to make the snap-fitting coupling possible.

There is further described a heart valve in which the coupling member is a peg. The peg can be provided on an elastic arm which extends from the structure of the central body and which in particular can be provided at the distal end of the elastic arm. The elastic arm can further be constructed in one piece with the structure of the central body; preferably, the peg can also be constructed in one piece with the central body, for example, by laser-cutting.

According to one aspect, there is described a prosthesis which comprises two or more sub-components which form the containment portion and corresponding two or more connecting blocks between the containment portion and the central body. This configuration may make use of a particularly advantageous variant, in which one of the connecting blocks is provided with at least one echo-opaque or radio-opaque marker. The marker or markers unambiguously identify the orientation of the prosthesis and also provide indications regarding the direction of the angular rotation to be imposed on the implant catheter for any potential corrections in terms of the orientation of the prosthesis. In this manner, another aid is provided for the operator who is implanting the prosthesis: by operating with transcatheter techniques, the operator can display the prosthesis during its positioning thereof only by means of echography or radiography. The limitations of these imaging methods, combined with a substantial symmetry in prosthesis configuration, make it particularly advantageous to have evident references for identifying the orientation of the prosthesis. Marker on one of the connecting blocks solves the problem.

According to another aspect, there is described a prosthesis in which the connecting arms are joined in pairs in the terminal area and each pair is provided with a single coupling mechanism.

According to another aspect, there is described a prosthesis having a connecting block which can be completely described as an intersection of two planar profiles which are projected at 90° with respect to each other. This feature makes the connecting block compatible with working techniques which involve a high level of quality and which are economically advantageous, while being capable of complying with all the functional and structural requirements associated with a transcatheter valve prosthesis described above. In particular it makes the construction thereof possible by means of a wire EDM process which ensures high dimensional precision, optimum quality of the surfaces and convenience in terms of production costs with respect to more conventional mechanical processing operations (for example, machining by chip removal) 4 components which are assembled by means of welding.

There is further described a method for constructing a connecting block for connecting a sub-component of a containment portion to a central body of a heart valve prosthesis for implantation with a transcatheter procedure. The method may comprise the step of providing a material slab. The method may comprise the step of carrying out, preferably by means of wire EDM, a first profiling along a first cutting path, obtaining a semi-finished product. The method may comprise the step of rotating the semi-finished product by a predefined angle. Preferably, the angle is 90°. The method may comprise the step of carrying out, preferably by means of wire EDM, a second profiling along a second cutting path, obtaining the connecting block.

### BRIEF DESCRIPTION OF THE DRAWINGS

Additional features and advantages will be appreciated from the following detailed description of a preferred embodiment of the invention with reference to the appended drawings which are provided purely by way of non-limiting example and in which:

FIG. 1 is a schematic view of the structural components of a prosthesis for a heart valve according to the invention, in an exploded configuration,

FIG. 2 shows the central body and a connecting block of the prosthesis for a heart valve of FIG. 1, in a state assembled with each other,

FIG. 3 is a perspective view of a connecting block between the central body and the containment portion, which is connected to a sub-component of the containment portion,

FIG. 4 is a front view of the connecting block of FIG. 3, connected to the central body,

FIG. 5 shows the central body according to a second embodiment,

FIGS. 6a and 6b are different perspective views of a connection mechanism between a connecting block according to the second embodiment and the central body shown in FIG. 5,

FIGS. 7a to 7d show the assembly procedure of the connecting block on the coupling mechanism according to the second embodiment shown in FIGS. 5 and 6,

FIGS. 8a to 8f show the steps of constructing the connecting block according to the second embodiment.

### DETAILED DESCRIPTION

Now with reference to the drawings, FIG. 1 shows an implantable heart prosthesis 10 which is used to replace the functionality of an atrioventricular valve.

The prosthesis 10 particularly comprises:
a central body 16,
a containment portion 18,
connecting blocks 20 for connecting the central body 16 and the containment portion 18,
a group of flexible prosthetic leaflets which are not depicted and which are fixed to the interior of the central body.

The prosthesis 10, similarly to each of the elements thereof, is configured so as to be collapsible without any negative effects on the safety and the functionality of the prosthesis itself. Therefore, it is possible to temporarily reduce the overall radial dimension of the central body 16 and the containment portion 18 in order to allow their introduction inside the heart cavities through accesses having small opening which are compatible with minimal invasive surgical techniques, with transcatheter techniques for the positioning and implantation of heart prostheses. In other words, it is possible to insert the heart prosthesis inside a catheter with a low radial profile which is capable of conveying the prosthesis through the cardiovascular system of the patient as far as the interior of the heart cavity, at the site of implantation, by means of an access with minimal invasiveness, and to carry out at that location the deployment and implantation thereof, functionally replacing the native valve.

In greater detail, the central body 16 is the portion of the prosthesis 10 which delimits the conduit for the passage of the blood through the device. The flexible prosthetic leaflets which make the blood flow unidirectional inside the conduit are fixed inside the central body 16, as known, for example, from the Italian Patent No. 20140204 from the same Applicant.

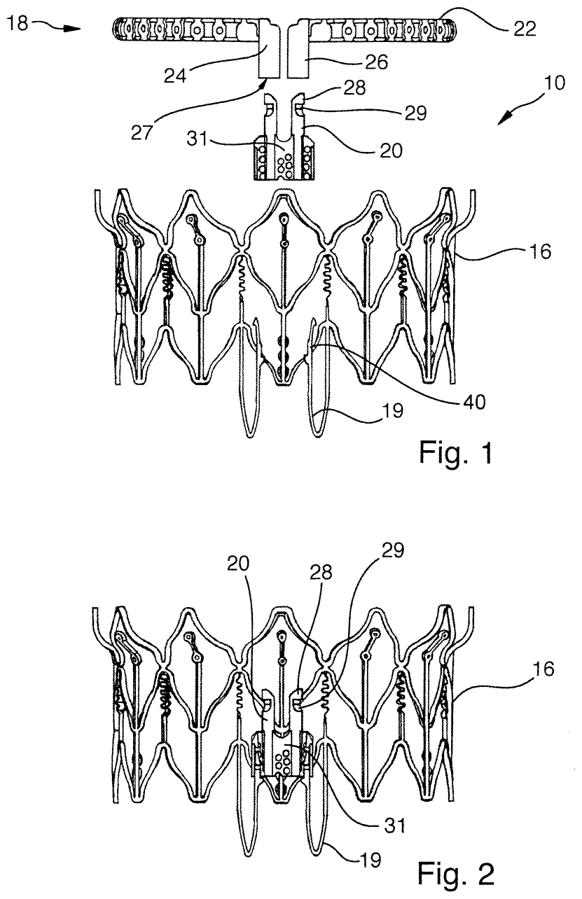

The central body 16 is an elastic structure which is radially collapsible and which tends, as a result of springback, to expand to a diameter even greater than the maximum diameter which maintains coaptation, that is to say the contact, among the free edges of the closed prosthetic leaflets. The central body is provided with connecting arms 19, to which the connecting blocks 20 are fixed, as can be seen in FIG. 2.

The containment portion 18 is the portion of the prosthesis 10 which opposes and limits the free expansion of the central body 16, preventing it from exceeding the maximum diameter which is compatible with the preservation of the coaptation between the prosthetic leaflets. The containment portion 18 has a substantially annular geometry and is longitudinally non-extensible, that is to say, it does not significantly modify the peripheral development thereof even when the central body 16 expands therein, by applying a radial force outwardly.

The containment portion 18 is preferably subdivided into two sub-components 22 which are separated from each other and which are substantially arcuate; for the sake of simplicity, the two sub-components will also be referred to below using the term “arcs”. Each arc 22 can be selectively coupled with the connecting blocks 20, to which it is then fixedly joined in the final implantation configuration. The Figures show only one of the two connecting blocks 20 but the embodiment described herein comprises two connecting blocks 20 which are positioned symmetrically relative to each other, one of which being concealed in the Figures by the visible connecting block 20.

Each arc 22 has two ends 24 and each of them is equipped with a coupling portion 26. The connecting blocks 20 are equipped with pins 28 which can be connected to the coupling portions 26. The pins 28 are connected to each other by a transverse structure 31 which is rigid and non-extensible. The function of this transverse structure 31 is to rigidly connect the ends 24 of the arcs 22, preventing two ends 24 of two contiguous arcs from being able to move apart from each other, with the prosthesis assembled, causing the inextensibility of the entire containment portion 18 to fail.

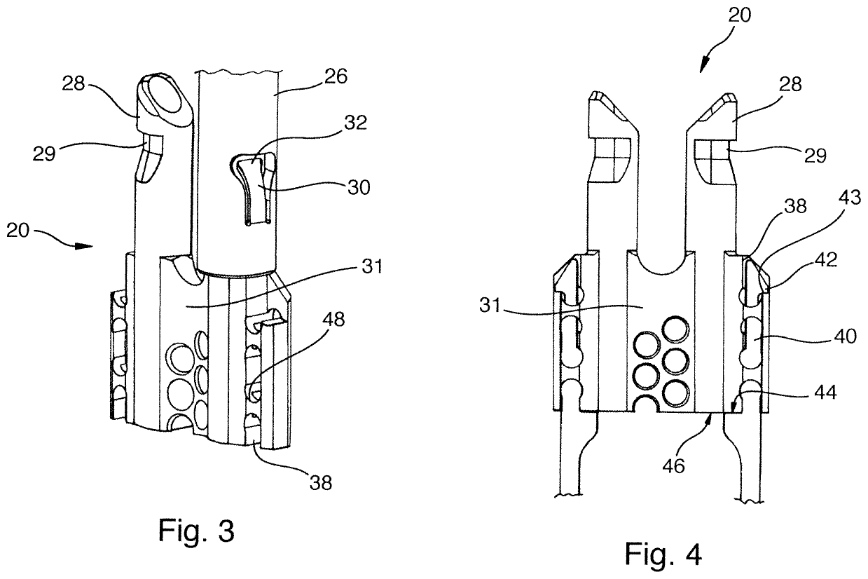

Now with reference to FIG. 3, in a preferred embodiment the pins 28 are provided with slots 29 on a lateral surface thereof. The coupling portions 26, at the ends 24 of the arcs 22, are substantially tubular and have axial holes 27 which are suitable for receiving the pins 28. The coupling portions 26 further have a cut-out in the tubular structure which defines a tongue 30. The tongue 30 is formed so as to extend towards the interior of the tubular structure so as to interfere with the slot 29 when the pin 28 is inserted in the axial hole 27. In fact, an end 32 of the tongue 30 moves into abutment with the slot 29 which is formed on the pin 28 and prevents the pin 28 from being extracted once inserted. It is thereby ensured that the arcs 22 cannot accidentally become separated from each other and from the connecting block 20, ensuring the stability of the prosthesis and therefore the safety of the patient. For the sake of clarity, the Figure shows only one coupling portion 26 of an arc 22 which is anchored in the respective pin 28 of the connecting block 20. During use, it will be understood that all the arcs 22 are fixed at the respective ends to the pins of the connecting blocks 20.

During use, following the connection between the arcs 22 and connecting blocks 20, the leaflets of the native valve remain trapped within the coupling between the central body 16 and the containment portion 18.

Now with reference to the detail of FIG. 4, the connecting blocks 20 have a coupling mechanism to the expansible central body 16.

This is particularly a coupling mechanism of the snap-fit type. In fact, the coupling mechanism comprises two housings 38 which are provided on each connecting block 20 for corresponding pegs 40 which are provided on the central body and in particular at the end of the connecting arms 19. The pegs are provided with a hooking tooth 42 for snapping onto an edge 43 which is formed in the relevant housing 38 on the connecting block 20.

The peg 40 is further provided with a stop surface 44 which is provided to move into abutment against the connecting block 20 and specifically against a lower face 46 thereof. In this manner, when the peg is inserted into the housing 38, it remains blocked in place, fixedly joined to the connecting block 20: the hooking tooth 42 prevents the peg from being extracted and the stop surface 44 prevents the peg from advancing further inside the housing 38.

In the embodiment shown in FIG. 4, the housing 38 is an elongate through-channel which extends in a direction which is substantially parallel with the direction in which the pins 28 of the connecting block 20 extend. The pins 28 and the pegs 40 are preferably located in the same plane when the connecting block 20 is fixed to the arm 19.

The housing 38 preferably provides for lateral openings 48 which make it easier for the blood to wash the interior of the housing 38.

As the Figures show, in the embodiment described there are provided two housings 38 for each connecting block 20, each of which is intended to receive a peg 40. The arms 19 in the embodiment depicted are paired in sets of two with respective pegs 40. An embodiment with a single arm 19, a single peg 40 and a single housing 38 provided on a connecting block 20 should not be excluded, but using at least two pegs and respective housings allows greater torsional stability of the mounted structure, which is particularly appreciated during the use thereof and particularly during the connection between the connecting block 20 and arcs 22, which is carried out during the implantation procedure of the prosthesis, directly in the ventricular chamber of the heart of the patient.

The pegs 40 are, as set out, preferably constructed at the ends of the arms 19 and can be constructed in one piece with the central body 16. It is particularly possible to construct the entire assembly formed by the central body, arms 19 and pegs 40 by means of laser-cutting directly from a tube of material with super-elastic behaviour, such as Nitinol.

In the embodiment shown in FIG. 4, the portion of the peg 40 positioned inside the housing 38 is characterized by two cross-sections with different dimensions. The cross-section near the stop surface 44 has dimensions very similar to those of the cross-section of the housing 38 in order to ensure the stability of the connection and therefore the stability of the joint between the connecting block 20 and arm 19. The cross-section of the peg 40 near the hooking tooth 42, instead, is under-dimensioned with respect to the cross-section of the housing 38, particularly in the direction in which the hooking tooth 42 is orientated, in order to allow the flexion of the end portion of the peg during the introduction of the hooking tooth 42 and the coupling thereof at the edge 43 of the housing itself. Naturally, the cross-section of the hooking tooth 42 is compatible with the insertion thereof through the housing 38.

The portion of the peg 40 near the hooking tooth 42 can also be preformed with a curvature counter to the deformation required for coupling the tooth 42 so as to make the coupling action thereof more stable because the peg 40 itself acts as a preloaded elastic element once it is inserted in place.

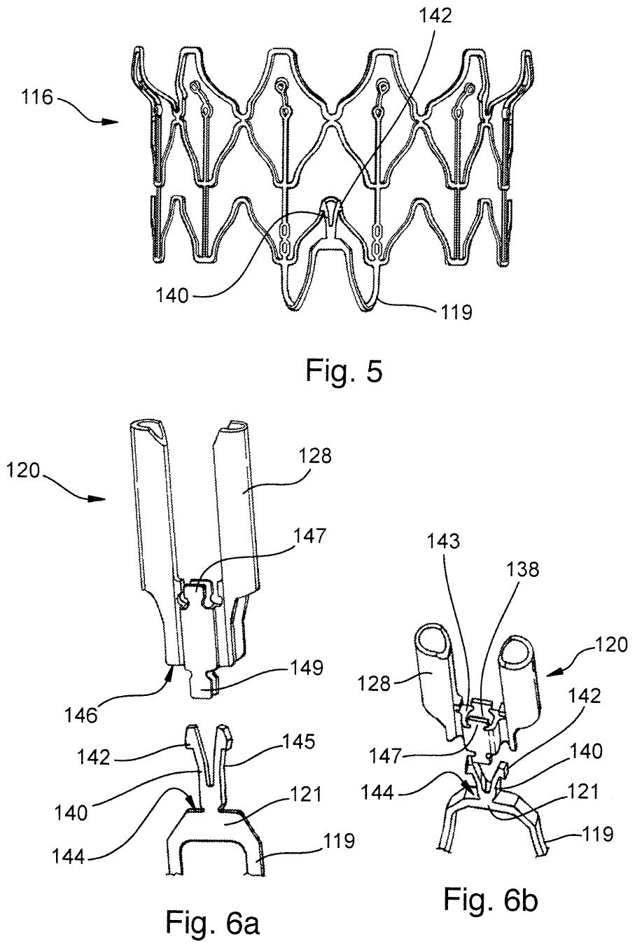

With reference to FIG. 5, in a second embodiment of the structure of the central body 116 of the prosthesis, the connecting arms 119 which grip on the same connecting block 120 are joined to each other at the respective ends in a joint portion 121. The coupling mechanism comprises a pair of contiguous pegs 140 which are positioned at the centre of the joint portion 121 between the two connecting arms 119. Both the pegs have a hooking tooth 142 at the end thereof, which teeth are orientated in opposite directions. In the version depicted, the pegs can have a configuration characterized by a predefined curvature 145 which is orientated in accordance with the orientation of the hooking tooth 142. The pegs 140 are therefore mutually divergent. In this manner, the coupling of the tooth is made even more stable as a result of the effect of the elastic preloading of the peg. In any case, it is not excluded that the pegs 140 may be straight, as are the pegs 40 in the embodiment in FIGS. 1 to 4.

FIGS. 6a and 6b show, according to two different projections, a connecting block 120 which is compatible with the central body 116 described in FIG. 5. In this specific embodiment, the connecting block 120 has a single housing 138 which is central and dimensioned so as to be able to receive both pegs 140. The coupling mechanism is therefore formed by a pair of pegs 140 and a common housing 138.

The hooking teeth 142 engage with the upper edge 143 of this housing 138. In this position, there are provided on the connecting block 120 two plates 147 which are preferably planar and which protrude from both sides of the housing 138. The plates mask the hooking teeth 142 in order to prevent a possible interference between them and the surrounding sub-valve structures of the atrioventricular valve (for example, the chordae tendineae). In fact, interference could result in damage to the anatomical structures themselves or even in a mechanical action on the hooking tooth which would cause disengagement thereof from the edge of the housing, with the risk of accidental separation of the block from the connecting arms. The joint portion 121 between the two connecting arms 119 also defines a stop surface 144 which, when the connecting block is coupled with the connecting arms, is in abutment with a lower face 146 of the connecting block. The interference between the hooking teeth 142 and the upper edge 143, in conjunction with the abutment between the stop surface 144 and the lower face 146 of the connecting block, axially stabilize the connecting block.

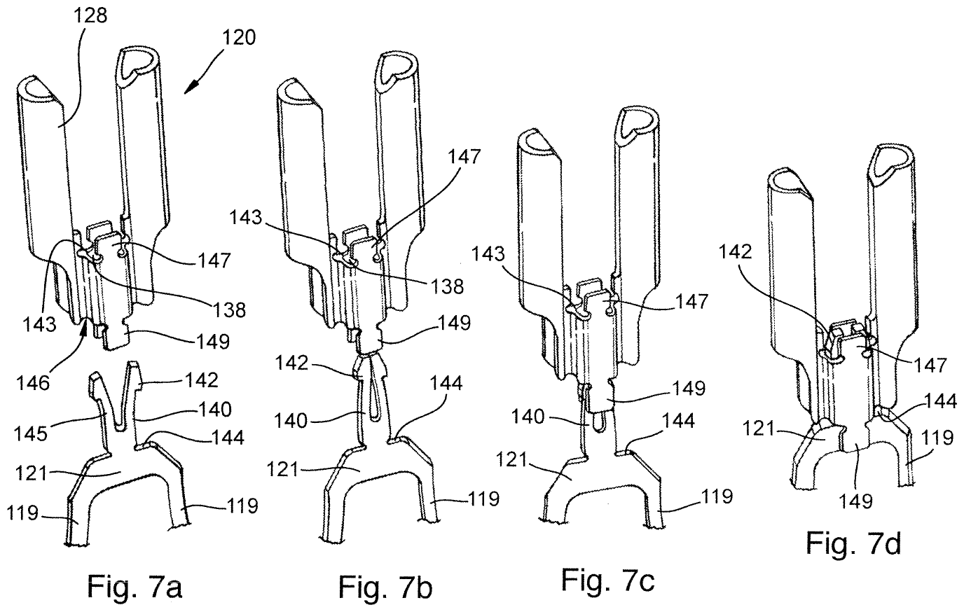

FIGS. 7a to 7d show the coupling procedure of the connecting block 120 with the connecting arms 119 according to the construction solution described in FIG. 6a. FIG. 7a shows the coupling mechanism with the pegs 140 in a non-deformed configuration. From this configuration, the pegs 140 are elastically deformed in order to move together until bringing into mutual contact the rear of the hooking teeth 142 (FIG. 7b). In this configuration, the complete front section of the hooking teeth 142 has a smaller spatial requirement than the cross-section of the housing 138 present on the connecting block 120. In this manner, the pegs 140 can be introduced into the housing 138 and can slide therein, as shown in FIG. 7c. Once the pegs 140 are completely inserted in the housing 138 of the connecting block 120 and the lower face 146 of the block has come into contact with the joint portion 121 between the connecting arms and particularly with the stop surface 144, as shown in FIG. 7d, the hooking teeth 142 can become separated from each other by means of springback of the pegs 140 and become engaged with the upper edge 143 of the housing 138.

In this manner, the connecting block 120 is fixedly joined to the connecting arms 119. The design of the connecting block can also provide for additional extension pieces, such as the two planar plates 149 which protrude from the lower surface of the block and which are engaged with the joint portion 121 between the two arms in order to provide greater stability for the connecting block in the face of lateral loads or torsional loads.

The connecting block 120 has pins 128 which are similar to the pins 28 described above for the first embodiment. The pins 128 allow the connection to the coupling portions 26 of the arcs 22.

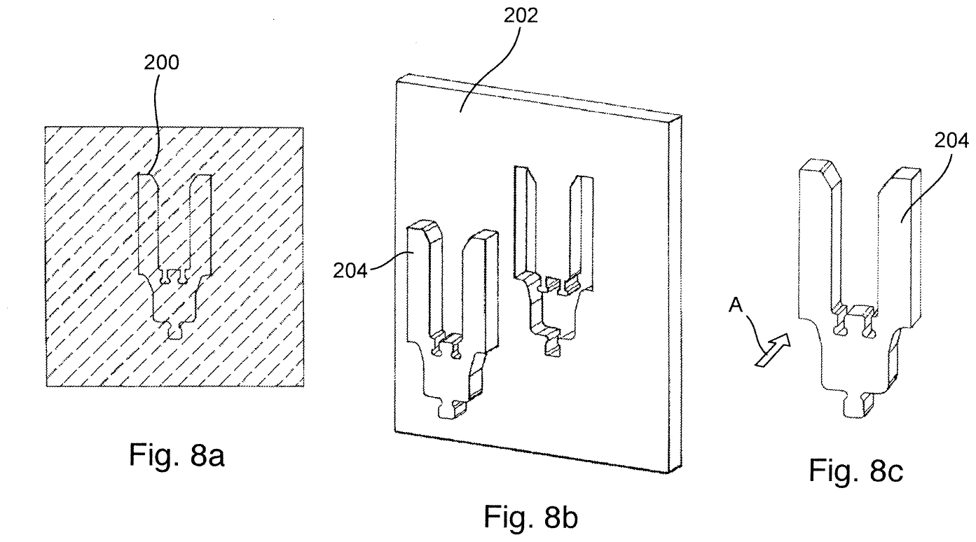

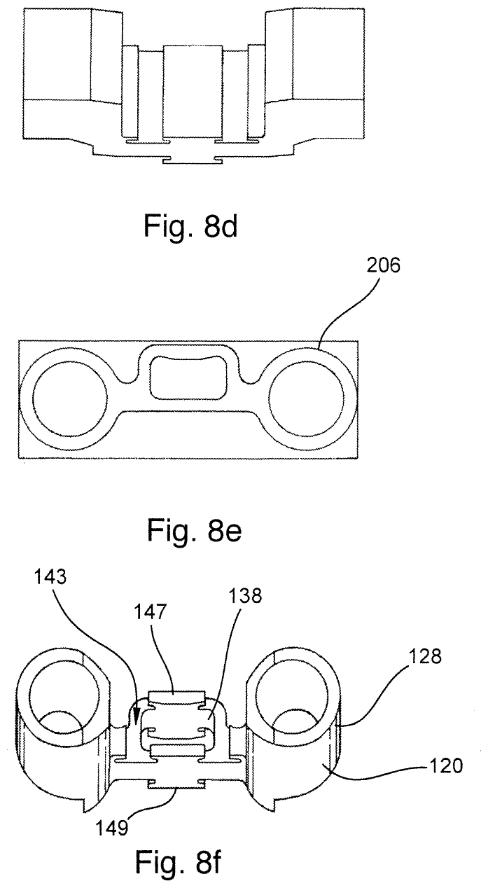

FIGS. 8a to 8f show, by way of non-limiting example, a process for constructing the connecting block 120 which requires only two processing steps with wire EDM, which technique allows low costs and high production volumes, while ensuring high standards of dimensional precision and quality of the surface finishes. FIGS. 8a and 8b show the first step of the processing operation, where a first wire cutting path 200, which can be seen in FIG. 8a and which is carried out on a slab 202 of the metal material selected for the connecting block 120, allows the construction of the prismatic semi-finished product 204 shown in FIG. 8c, already having the same profile as the final component, when viewed in a direction A. By rotating the prismatic semi-finished product 204 by 90°, according to the perspective indicated in FIG. 8d, it is possible to carry out the second step of the processing operation, carrying out a second cutting path along the profile 206 described in FIG. 8e. This last profile allows the final geometry of the connecting block 120 to be obtained, as shown in FIG. 8f, without any for additional subsequent processing operations.

It may be noted that the production method described above is allowed as a result of the effect of the specific design of the connecting block 120 which can be completely described as an intersection of two planar profiles which project at a precise angle with respect to each other.

An additional advantage of the embodiments of the invention described above involves the reversibility of the coupling mechanism between the connecting block 20, 120 and the connecting arms 19, 119. In fact, it is possible, by means of appropriate mechanical tools, to disengage the hooking tooth or teeth 42, 142 from the edge 43, 143 of the housing 38, 138 in order to be able to remove the peg 40, 140 from the housing itself and therefore to separate the connecting block from the connecting arms.

Reversibility is to be understood here as the possibility of removal of the connecting block from the connecting arms during the assembly phase in the production cycle of the prosthesis, or at any stage prior to insertion of the prosthesis into the patient's body. The coupling mechanism is, however, configured to be irreversible during the phase of insertion into the patient's body and then during use, i.e. when the valve is implanted. The connection between the components is in fact stable under operating conditions and the connecting block cannot in any way accidentally disengage from the connecting arms. Accidental release would in fact result in separation between the containment portion and the central expandable portion, leading to malfunctioning of the prosthesis, or even migration of the prosthesis, with harmful outcomes for the patient. In the configuration shown in the drawings, the hooking teeth 42, 142 are set back with respect to the thickness of the wall defining the upper edge 43, 143 on which the hooking teeth 42, 142 engage. They thus remain protected and cannot accidentally disengage.

It is understood that arrangements other than this may be envisaged to ensure that the connection is stable. By way of example only, the pegs 40, 140 can be made rigid enough to exclude, in view of the forces and stresses involved during insertion into the patient's body and during use, that they can bend and thus cause the coupling mechanism to disengage. Alternatively, locking devices can be provided to prevent the release of the attachment mechanism.

According to a variant which is not depicted, one of the connecting blocks 20, 120 can be provided with a radio-opaque or echo-opaque marker which allows it to be identified with absolute certainty by means of the imaging instruments (echocardiography and radiography) usually used in the transcatheter procedures.

The above-described embodiments always make reference to the presence of two connecting blocks, equal to the number of sub-components 22 of the containment portion in the configuration of the prosthesis of FIG. 1. Naturally, the prosthesis may also comprise a different number of sub-components 22 of the containment portion. The version described, with two sub-components 22, is the preferred one because it allows the use of two guide wires which, as described in the patent application WO 2021/014400, are easier to position correctly than a single guide wire which could remain entangled in the chordae tendineae. However, it should not be excluded that the containment portion may comprise a single sub-component and may therefore be formed in the manner of a cut ring. In this case, a single connecting block will be provided.

Finally, there should also not be excluded the solution which has a third sub-component, notwithstanding the greater complexity required by the positioning operations, mainly for a prosthesis which is intended for replacing the tricuspid heart valve. If there are provided three or more sub-components and therefore three or more connecting blocks, it may be particularly advantageous to provide radio-opaque or echo-opaque markers, which may also be different from each other, in two of the three connecting blocks.

It should further be underlined that the coupling mechanism between the central body 16, 116 and the connecting block 20, 120 which is described above and which is formed by housings 38, 138 and pegs 40, 140 may also be used in a connecting block which has a different connection system with respect to the arcs 22. Similarly, the pin/hole connection mechanism between arcs 22 and pins 28, 128 described above can also be used with connecting blocks 20, 120 which have a different coupling system with respect to the central body 16, 116.

Naturally, the principle of the invention remaining the same, the forms of embodiment and details of construction may be varied widely with respect to those described and illustrated without thereby departing from the scope of the invention.

1. A prosthesis for a heart valve for implantation with a transcatheter procedure comprising an expansible central body, a containment portion having one or more sub-components, and at least one connecting block separate from the central body for connecting each sub-component of the containment portion to the central body, wherein for the at least one connecting block there is provided a mechanical coupling mechanism (for coupling the at least one connecting block with the expansible central body.

2. The prosthesis for a heart valve according to claim 1, wherein the coupling mechanism comprises a snap-fit mechanism.

3. The prosthesis for a heart valve according to claim 2, wherein the coupling mechanism comprises at least one housing provided on the at least one connecting block and at least one corresponding coupling member provided on the central body, the at least one corresponding coupling member being is inserted into the at least one housing provided on the at least one connecting block, the at least one corresponding coupling member including a hooking tooth for coupling in a snap-fitting manner in the at least one connecting block.

4. The prosthesis for a heart valve according to claim 1, wherein the coupling mechanism includes an abutment element configured to move into abutment against the at least one connecting block.

5. The prosthesis for a heart valve according to claim 3, wherein the at least one connecting block comprises two pins engageable in corresponding holes provided in the one or more sub-components of the containment portion and the at least one housing is elongate in a direction substantially parallel with axes of the pins.

6. The prosthesis for a heart valve according to claim 2, wherein the coupling mechanism comprises a coupling member, the coupling member comprising a peg provided at a distal end of an elastic arm, the elastic arm being constructed in one piece with the central body.

7. The prosthesis for a heart valve according to claim 1, wherein the at least one connecting block comprises two connecting blocks, one of the two connecting blocks being provided with a radio-opaque or echo-opaque marker.

8. The prosthesis for a heart valve according to claim 1, wherein the at least one connecting block comprises two pins engageable in corresponding holes provided in the one or more sub-components of the containment portion and a transverse structure connecting the two pins, each pin being provided with a slot for allowing the one or more sub-components of the containment portion to be engaged in a snap-fitting manner.

9. A method for constructing a connecting block for connecting a sub-component of a containment portion to a central body of a prosthesis for a heart valve for implantation with a transcatheter procedure, comprising the steps of:
providing a slab of material;
carrying out a first incision along a first cutting path, and obtaining a semi-finished product;
rotating the semi-finished product by a predefined angle; and
carrying out a second incision along a second cutting path, and obtaining the connecting block.

10. A method for constructing a heart valve prosthesis for implantation by transcatheter procedure including an expandable central body, a containment portion having one or more sub-components, and at least one connecting block for connecting each sub-component of the containment portion to the central body, the method comprising the steps of:
providing a slab of material;
carrying out a first incision along a first cutting path, and obtaining a semi-finished product;
rotating the semi-finished product by a predefined angle; and
carrying out a second incision along a second cutting path, and obtaining the connecting block; and
connceting the connecting block to the central body.

11. The method according to claim 9, wherein the step of carrying out the first incision along the first cutting path is performed by wire EDM and the step of carrying out the second incision along the second cutting path is carried out by wire EDM.

12. The method according to claim 10, wherein the step of carrying out the first incision along the first cutting path is performed by wire EDM and the step of carrying out the second incision along the second cutting path is carried out by wire EDM.
